(19) United States
(12) Patent Application Publication
(10) Pub. No.: US 2026/0193598 A1
(43) Pub. Date: Jul. 9, 2026

(54) FERMENTED MATERIAL COMPRISING STALK PARTICLES OF MONOCOTYLEDONOUS FLOWERING PLANTS, AND ITS PRODUCTION METHOD

(71) Applicant: CORMO AG, St-Aubin (CH)

(72) Inventors: Stefan GRASS, St-Aubin (CH); Matthias THALER, St-Aubin (CH); Jonas LECHOT, St-Aubin (CH)

(73) Assignee: CORMO AG, St-Aubin (CH)

(21) Appl. No.: 19/131,750

(22) PCT Filed: Nov. 22, 2022
(86) PCT No.: PCT/EP2022/082867

§ 371 Date: May 21, 2025

(51) Int. Cl. C12N 5/00; A01G 24/22

(52) U.S. Cl. CPC C12N 5/0018; A01G 24/22

(57) ABSTRACT

A fermented material included stalk particles of monocotyledonous flowering plants which can constitute a sustainable, ecofriendly & bio-sourced material. More particularly, the fermented material includes stalk particles of monocotyledonous flowering plants and at least one filler (clay), wherein: (a) the particles have a size given by their greatest dimension D^{g}, in mm such as: D^{g}≤30; (b) the specific density D^{s} of the material, in g dry matter per liter such as 100≤D^{s}≤250; (c) the specific water content C^{w} of the material is, in % by mass such as 55≤C^{w}≤95; and the carbon/nitrogen ratio C/N of the material is such as 60≤C/N. A method is provided for the production of this fermented material.

### TECHNICAL FIELD

The invention relates to new bio-sourced materials, especially to a fermented material comprising stalk particles of monocotyledonous flowering plants, preferably maize.

This fermented material can be a culture medium and/or plant substrate component and/or a peat substitute, notably a casing material.

The implementation of said new bio-sourced materials in their numerous uses, as well as the preparation of the products/compositions derived from said new bio-sourced materials, are encompassed in the present invention.

The invention also concerns a method for the production of said fermented material.

### BACKGROUND ART

U.S. Pat. No. 10,260,169B2 describes a method for the production notably of superabsorbent pellets and/or of a fibrous material from crop residues of monocotyledonous flowering plants cultivation, comprising the following steps:
(i) cutting the maize stalks planted in the ground of the field below the lowest cob of the stalks, so that leafy stalk segments stay on the field; each less leafy stalk segment including a spongy core, a stalk bark wrapping the core and a leafy matter wrapping the stalk bark or born by the stalk;
(ii) cutting the less leafy stalk segments as close to the ground as possible;
(iii) harvesting the less leafy stalk segments cut in step (ii);
(iv) cutting the less leafy stalk segments harvested in step (iii) into stalk sections which largest dimension in mm is comprised between 5-50;
(v) providing a mechanical impact to the stalk sections of step (iv) so as to separate spongy cores from stalk barks, as well as the leafy matter, to transform said stalk barks into elongated fiber pieces; and to obtain a mix containing:
f1. said spongy cores forming a superabsorbent pellets fraction which water absorption capacity expressed in multiple of its own dry mass is greater than or equal to 15;
f2. said elongated fiber pieces forming a fibrous matter fraction;
f3, and said leaf matter forming a leafy fraction, the superabsorbent pellets fraction, the fibrous matter fraction, and the leafy fraction forming three fractions;
(vi) separating the three fractions from each other;
vii) recovering the three fractions f1-f2-f3;
(viii) optionally reducing the f1 superabsorbent pellets up to a smaller pellet size to have a largest dimension in mm between 0.1-20 mm.

The U.S. Pat. No. 10,260, 169B2 superabsorbent pellets f1 and fibrous material f2 are prime ingredients for the preparation of different compositions which are intending to be used in different fields: chemical treatments, depollution, purification, filtration, industrial processing aid, crop activation. In particular, fibrous matter f2 can be used as a plant substrate, plant substrate component and/or peat substitute.

The U.S. Pat. No. 10,260, 169B2 fibrous material f2 are improvable as plant substrate, plant substrate component and/or peat substitute, especially regarding the growing efficiency and/or the growing selectivity of certain kind of plants or fungi.

Casing soil is currently produced almost exclusively from peat, which is a fossil resource. Thus, the production of mushrooms in its present way is not sustainable. Alternative raw materials for the production of casing soil have been tested for a long time (e.g. grass, wood fibers, waste paper, cocopeat), but they do not meet the very specific requirements of this application (structure formation, water holding capacity, biological stability) and are therefore not relevant in practice today.

WO2012066511A1 discloses a method for preparing casing material (pH of 7.5) for button mushroom production includes the steps of:
providing a body of plant parenchyma tissue or pith extracted from a plant source in the form of sugarcane bagasse;
mixing the body with water to provide a starting mixture;
adding a decomposition enhancer (cattle rumen content), to the starting mixture for stimulating microbial activity;
allowing the starting mixture to compost; and
allowing excess nutrients to leach from the mixture.

Watering of the fermented material is carried out on days 1 2, 7, 12, 17, 22, 27, 32, 37, 42, 47, 52, 57, 62, 67, 72 and 77 of composting, with the aim of maintaining the moisture content of the composting casing material at 65%, throughout the composting period. During the composting stage, care should be taken not to allow the composting casing material to dry out, as it will inhibit microorganism growth and composting. The ratio of carbon to nitrogen (C:N) of the casing material is maintained at 40-50:1.

The casing material has a water holding capacity in excess of 350 ml water/100 g dry mass. The casing is used in a method of producing button mushrooms {Agaricus bisporus).

The casing material according to WO2012066511A1 has the following drawbacks: the low C:N ratio results in poor biological stability and the cattle rumen content adds undesirable salts.

In this context, the design of new bio-sourced materials that takes into account the environmental concern, especially, but not only, with respect to the spoiling of to-day preserved peat bogs, is a necessity of public order. These new bio-sourced materials must not only be environment friendly but must also perform well in terms of productivity (culture yield for growing media), crop quality, and crop selectivity. It is notably true for casing material as peat substitute.

It is suitable these new bio-sourced materials, as culture media, have an improved water holding capacity and/or biological stability and/or a low content of nitrogen and/or phosphorous and/or sulfate.

It exists also a need to have the fields rapidly cleared from the stalks/stems, after grains harvest, especially for maize. Said need is linked to the necessity of having crop rotation and of preventing the decomposition of the remaining vegetal material after grains harvest, which can be a source of pollution of groundwater, as well as controlling crop infestations, notably the corn stem borer.

Another concern in the crop field is the biological stability and storage safety of the remaining vegetal material, after grains harvest. The stalks/stems below the cob attachment are significantly wetter than the grains and have a dry matter content of only 25-30%, depending on weather conditions. At this moisture content, rapid development of microorganisms associated with material degradation and heat generation can be observed on a central raw material store. The material degradation leads to an undesired material loss and the heat development results in a fire risk. This situation makes the storage of wet corn stalks for subsequent utilisation impossible.

### OBJECTIVES OF THE INVENTION

In this context, the invention aims to satisfy at least one of the following objectives.

An objective of the invention is to provide a fermented material comprising stalk particles of monocotyledonous flowering plants, preferably maize, which can constitute a sustainable, ecofriendly & bio-sourced material.

An objective of the invention is to provide a fermented material comprising stalk particles of monocotyledonous flowering plants, preferably maize, which can constitute a sustainable ecofriendly bio-sourced material, useful as culture medium and/or plant substrate component and/or fertilizer and/or a peat substitute, notably for casing.

An objective of the invention is to provide a fermented material comprising stalk particles of monocotyledonous flowering plants, preferably maize, which can constitute a sustainable, ecofriendly & bio-sourced material, useful as high-performance and/or selective culture medium and/or plant substrate component and/or fertilizer and/or a peat substitute, notably for casing.

An objective of the invention is to provide a fermented material comprising stalk particles of monocotyledonous flowering plants, preferably maize, which can constitute a sustainable, ecofriendly & bio-sourced material, useful as culture medium and/or plant substrate component and/or fertilizer and/or a peat substitute, notably for casing, and having a high-water holding capacity, and/or a biological stability and/or a low content of nitrogen and/or phosphorous and/or sulfate.

An objective of the invention is to provide a method for the production of fermented material comprising stalk particles of monocotyledonous flowering plants, preferably maize, which can constitute a sustainable, ecofriendly & bio-sourced material, said method being simple and industrial.

An objective of the invention is to provide a method for the production of fermented material comprising stalk particles of monocotyledonous flowering plants, preferably maize, which can constitute a sustainable, ecofriendly & bio-sourced material, said method being economical and swift.

An objective of the invention is to provide a method for the production of fermented material comprising stalk particles of monocotyledonous flowering plants, preferably maize, which can constitute a sustainable, ecofriendly & bio-sourced material, said method making possible the removal from the fields, of the remaining vegetal material after grains harvest.

An objective of the invention is to provide a method for the production of fermented material comprising stalk particles of monocotyledonous flowering plants, preferably maize, which can constitute a sustainable, ecofriendly & bio-sourced material, said method addressing the problem of the moisture content (70-75% depending on weather conditions) of the remaining vegetal material (stalks) after grains harvest, and of the subsequent difficulty of biological stability and storage of said remaining vegetal material.

### SUMMARY OF THE INVENTION

The inventors have had the merit to invest a lot of time and money in R&D to eventually find out a new crop bio-sourced material, complying with at least one of the above objectives. This is how the invention concerns a fermented material comprising stalk particles of monocotyledonous flowering plants, preferably maize, and, possibly at least one filler/additive, preferably at least one clay, wherein:
(a) the particles have a size given by their greatest dimension D^{g}, in mm and in an increasing order of preference, such as:

$${D}^{g} \leq 30 ; {D}^{g} \leq 20 ; {D}^{g} \leq 15 ; {D}^{g} \leq 10 ; {D}^{g} \leq 5 ;$$

(b) the specific density D^{s} of the material, in g dry matter per liter and in an increasing order of preference, such as:

$$100 \leq {D}^{s} \leq 200 ; 120 \leq {D}^{s} \leq 190 ; 130 \leq {D}^{s} \leq 180 ; 140 \leq {D}^{s} \leq 180 ;$$

(c) the specific water content CW at saturation of the material is, in % by mass and in an increasing order of preference, such as:

$$55 \leq {C}^{w} \leq 95 ; 65 \leq {C}^{w} ; 70 \leq {C}^{w} \leq 90 ; 80 \leq {C}^{w} \leq 85$$

(d) the Carbon/Nitrogen ratio C/N of the material is, in an increasing order of preference, such as:

$$60 \leq C / N ; 70 \leq C / N ; 80 \leq C / N ; 90 \leq C / N ; 95 \leq C / N \leq 120 .$$

The eco-friendly and crop-sourced material according to the invention is a noteworthy new raw material, notably useful as culture medium and/or plant substrate component and/or fertilizer and/or a peat substitute, notably for casing, especially for mushrooms production, such as button mushrooms. This fermented material according to the invention has a peculiar fibrous and porous structure. It has an extraordinarily high-water retention capacity. This is achieved by not only utilizing the matric binding capacity of the fibers (i.e. the ability of the cellulose fibers to absorb and retain water using hydrogen bonds), but also by developing a capillary binding capacity (the formation of fine pores filled with water). Through its porosity, the material shrinks to a smaller volume with the addition of water, and develops capillary binding forces and form water-filled agglomerates (lumps), exactly as it is suitable, notably in the production of casing soil.

In order to be a culture substrate of choice, notably as casing soil for the mushrooms production, the fermented material has to comply with a key specification, which is pretty antinomic with its high-water retention capacity, namely a biological activity at the lowest possible level to prevent the unwanted propagation of foreign organisms.

Moreover, the fermented material according to the invention is advantageous thanks to its capacity to be agglomerated and so to be shapable in various 3D forms, having a certain mechanical strength.

Preferably, the fermented material according to the invention is characterized in that at least a part of the particles are fibers particles, the average length L^{a} of the fibers particles being preferably, in mm and in an increasing order of preference, such as:

$$0.5 \leq {L}^{a} \leq 3 ; 0.6 \leq {L}^{a} \leq 2.5 ; 0.7 \leq {L}^{a} \leq 2 ; 0.8 \leq {L}^{a} \leq 1.8 ; 1 \leq {L}^{a} \leq 1.5$$

Advantageously, the fermented material according to the invention has a biological stability corresponding to an oxygen consumption O^{c} lower than or equal, in g per day per g of material, and in an increasing order of preference: 3; 2.8; 2.7; 2.6; 2.5; 2.4; 2.3; 2.2; 2.1; 2.

In preferred embodiments, the fermented material according to the invention is a culture medium and/or plant substrate component and/or fertilizer and/or a peat substitute, like a casing soil for instance.

The fermented material according to the invention can be mixed with standard casings in a proportion preferably of up to 70% v/v, preferably 5-40% v/v.

The invention is also directed to a method for the production of the fermented material according to the invention, comprising the following steps:
.S1. harvesting the grains of monocotyledonous flowering plants, preferably from the subfamily Panicoideae, and more preferably maize, so as the stalks remain stand on the field;
.S2. cutting and shredding the remaining stems/stalks standing on the field into a particulate material;
.S3. possible transporting the particulate material to a processing site;
.S4. possible dewatering the particulate material up to a dry matter content Cam, in % by mass and in an increasing order of preference, such as:

$$20 \leq {C}^{d m} ; 25 \leq {C}^{d m} \leq 60 ; 30 \leq {C}^{d m} \leq 50$$

.S5. storing and submitting the particulate material, to a fermentation, preferably during a time sufficient so that the temperature T^{p} of the stored particulate material reaches a value around 50° C., and the total dry matter of the particulate material has a variation percentage per 24H, equal to or lower than, in an increasing order of preference, 20%; 15%; 10%; 5%; 1%;
.S6. grinding the particulate material up to a particle size given by their greatest dimension D^{g}, in mm and in an increasing order of preference, such as:

$${D}^{g} \leq 30 ; {D}^{g} \leq 20 ; {D}^{g} \leq 15 ; {D}^{g} \leq 10 ; {D}^{g} \leq 5$$

.S7. water spraying on the grinded particulate material from (S6);
.S8. possible dewatering the grinded particulate material, so as to get a dry matter content Cam, in % by mass, such as:
   C^{dm}≤37, preferably C^{dm}≤35, and more preferably 20≤C^{dm}≤35;
.S9. loosening of the particulate material;
.S10. possible addition of at least one filler/additive, preferably consisting in at least one clay;
.S11. possible submitting the particulate material, to a fermentation during a time sufficient, so that the temperature of the fermented particulate material reaches a value T^{p} (° C.) greater than or equal to, in an increasing order of preference; 50; 55; 60; 65;
.S12. possible maintaining the fermented particulate material at the temperature T^{p} during at least, in hours and according to an increasing order of preference, 12; 24; 36; 48;
.S13. possible storing and/or conditioning the fermented particulate material from .S12.

This efficient, industrial, and reliable method leads notably to the advantageous fermented material according to the invention, by recovering rapidly, less than 2-3 weeks, after the grains harvesting, the stalks of the plant, and by transforming them into a technical and functional fermented growing raw material.

This method respects all requirements of authorities and raw material suppliers, and especially regulations supporting groundwater protection and the control of the corn stem borer, which migrates in the corn stalks to the soil after harvest, overwinters there and causes crop damage in the following year.

This method does not allow rapid development of microorganisms associated with material degradation and heat generation, during storage of the recovered crop material. It means undesired material loss does not occur, as well as the control of heat development according to this method significantly limits the fire risk during bulk storage.

According to a variant of this method for the production of the invention fermented material, the starting products are not the stalks are not directly extracted from crop fields and shredded in situ, but cut stalks stored in suitable conditions in this state.

This variant corresponds to the method described in § wherein the steps .S1., .S2. & .S3. are replaced by the following steps:
.S01. implementing cut stalks of monocotyledonous flowering plants, preferably from the subfamily Panicoideae, and more preferably maize;
.S02. shredding the cut stalks from (S01) into a particulate material;
.S03. possible storing of the particulate material.

The method according to the invention has also at least one of the following features: further concerns.
(—f.1—): step (S2) is implemented by means of forage harvester.
(—f.2—): step (S2) takes place within 20 days, preferably within 14 days after the grain harvest.
(—f.3—): step (S02) is implemented by means of a forage harvester.
(—f.4—): step (S2) or (S02) leads to stalk particles having a particle size given by their greatest dimension D₂^{g}, in mm and in an increasing order of preference, such as:

$${D}_{{2}^{g}} \leq 15 ; {D}_{{2}^{g}} \leq 12 ; {D}_{{2}^{g}} \leq 9 ; {D}_{{2}^{g}} \leq 6 ; {D}_{{2}^{g}} \leq 3$$

(—f.5—): step (S4) is a mechanical dewatering implemented by means of a screw press
(—f.6—):
   step (S5) of composting consists in storing the dewatered particulate material from (S4) or (S04) in an aerated and insulated composting container;
   step (S11) of composting consists in storing the particulate material in an aerated and insulated composting container.
(—f.7—): the aerated and insulated composting container of (—f.5—) is a modified refrigerated shipping container.
(—f.8—): step (S6) is a grinding implemented by means of an impact mill or of a grinding mill, preferably by means of an impact mill and more preferably by means of an impact mill which tip speed of the rotating working tools is greater than or equal to, in m/s and in an increasing order of preference; 60; 65; 70; 75; and more preferably comprised between 80 and 100.
(—f.9—): in step (S7), the addition of water is comprised between 100 and 200 L/m³, preferably between 130 and 170 L/m³, and more preferably between 145 and 155 L/m³.
(—f.10—): in step (S10), the addition of filler/additive is comprised between 5 and 45 kg/m³, preferably between 15 and 35 kg/m³, and more preferably between 20 and 30 kg/m³.
(—f.11—): in step (S10), the filler is chosen in the group comprising-ideally consisting of-clay, peat and mixtures thereof.

### Definitions

According to the terminology of this text, the following non limitative definitions have to be taken into consideration:
any singular is equivalent to a plural.
“around” means for instance more or less 10%.
“stalk” stands for “stem” and reciprocally.
“peat” refers to organic material extracted from peat bogs.
“casing” refers to the top layer of mushrooms growing substrate, especially Agaricus bisporus growing substrate.
“compost” refers to the lower layer and nutrient source for mushrooms, especially for Agaricus bisporus.

### DETAILED DESCRIPTION

### Fermented Material

The crop-sourced material according to the invention comprises particles of monocotyledonous flowering plants, preferably maize. These particles result from the cutting, the shredding and the grinding of stalks, preferably maize stalks.

This particulate material has preferably at least one of the following characteristics:
(a) Greatest dimension D^{g}≤1 mm. The greatest dimension D^{g} of the material can be determined as described in detail hereafter: 100 g of the material are dried at 105° C. The material is than passed through a sieve with a mesh of 1.5 mm. The percentage of passage defines the fiber dimension, e.g. >30%; >40%; >50%. The preferred greatest dimension corresponds to a passage of around 50%.
(b) Specific density 100≤D^{s}≤180 g dry matter/L per liter and advantageously 120≤D^{s}≤160 g dry matter/L. D^{s} is preferably around 140 g dry matter/L and can be determined as described in detail hereinafter: A big bag of 1000 L volume is filled with fermented material and weighed. Five samples of around 0.5 L each are taken and blended for creation of one representative sample. 100 g of this sample are dried to constant mass at 110° C. The dry matter content is the dry mass in % of the wet sample mass (100 g). Specific density D^{s} is calculated by multiplication of the fresh mass in the big bag by dry matter content.
(c) Specific water content CW at saturation, i.e at the saturation point which is the maximum water content a given material volume can hold without allowing any leakage of liquid. The saturation point was measured using a fine sieve with an open mesh of 0.5 mm, filling it with 100 g of material, draining it with water, and waiting until no more leakage occurred, respectively no more water was passing through the sieve.

C^{w} can be determined as described in detail hereinafter: First, the dry matter content of a defined quantity (in grams) of crop-sourced & fermented material according to the invention is determined according to the procedure defined above. Second, the saturation point of that material was measured according to the procedure defined above. The specific water content in g/l is calculated as (total mass at saturation point in g/l)−(dry mass in g/l).

According to the invention C^{w} is so defined:

70≤C^{w}≤90% by mass and advantageously 75≤C^{w}≤85% by mass. The specific water content at saturation C^{w} is preferably around 80% by mass and
(d) 95≤C/N≤120. (“C” for carbon atom and “N” for nitrogen atom). C/N is determined using the following standard procedure https://en.wikipedia.org/wiki/Carbon-to-nitrogen ratio. The fermented material is burned to ash at 950° C. for release of the CO₂ and N₂ contained therein, detection of C and N in the gas stream and expression as total mg C per g of material and total mg N per g of material, and calculation of the ratio C/N.

These particles are at least partly fibers particles, especially cellulosic fibers particles which form a porous network included in a matrix made of fermented crop material and possibly containing at least one filler: clay for instance. Other fillers like peat can be contemplated.

The fiber fineness, i.e. the average fiber length and fiber length distribution are important features of the fermented material according to the invention. Indeed, the fiber length distribution is expected, on the one hand, to favour the formation of very fine pores and, on the other hand, to contain sufficiently long fibers which favor the coherence of the agglomerates which can be formed with the material, and thus the strength of these agglomerates. According to a best embodiment of the invention, an average fiber length of 1-1.5 mm give the best results.

The biological stability is another way to define the fermented material according to the invention. So, said material has a biological stability corresponding to an oxygen consumption Oc lower than or equal, in g per day per g of material, and in an increasing order of preference: 3; 2.8; 2.7; 2.6; 2.5; 2.4; 2.3; 2.2; 2.1; 2.

The oxygen consumption OG can be determined as described in detail hereinafter: For every atom of carbon (C) one molecule of oxygen (O₂) is consumed in the oxidation reaction. Dry matter loss during composting in the container is directly linked to oxygen consumption. Total fresh mass and their respective dry matter contents are measured (see § [0041]) before and after the composting process. The difference in total dry matter multiplied by 1.14 yields the oxygen consumption for the entire container. Cellulose which is the main organic component in maize straw has a molar carbon to oxygen ratio of 1:1. During biological degradation, one oxygen atom from air and one O atom from cellulose are required to remove one C. Biological degradation is limited by the availability of nutrients and water. If they are consumed, the oxygen consumption goes down and the degradation comes to a halt.

The fermented material according to the invention can be mixed with standard casings like dark milled peat, dry peat or semi-dry peat. The proportion of standard casing to fermented material in such mixture can be up to 70% v/v, preferably 5-40% v/v, 20% v/v for instance. Such a standard casing incorporation can be useful to buffer the fermented material according to the invention and/or to reduce the electrical conductivity and pH of the mixture to e.g. <1.2 mS/dm and <7.5, respectively, which may also favour fungal growth.

### Method for the Production Notably of the Fermented Material According to the Invention

### Main Mode of Implementation: Steps S1 to S13

Step S1: Harvest of the grains using a standard corn thresher, with an elevated cutting bar, leaving stalks with a height of e.g. 50-100 cm standing on the field. Grain harvesting is accomplished for instance using a John Deere type corn thresher with a raised cutter bar. A reduced-width caterpillar drive is used on the thresher to prevent harvest losses.
Step S2: Cutting and, preferably shredding, of the remaining stems/talks in the field, using a forage harvester, with a cutting length of e.g. 2-8 mm to get a chopped particulate material. S2 is implemented for instance less than 10 days after step S2, complying so with the requirements of authorities and raw material suppliers. In particular, it is performed within 2 hours after grain harvesting using a chopper, type Claas Jaguar, with a theoretical chop length of 6 mm.
Step S3: Possible transportation of the chopped particulate material from S2 by any conventional means.
Step S4: Possible dewatering of the chopped particulate material from S2 with a screw press to get a pressed cake having a dry matter content C^{dm} of e.g. 40-45%. For instance, S4 is carried out within a maximum of 2 weeks after S2 with a dewatering press, type Trumag®, with a final moisture content of 56%, corresponding to a dry matter content C^{dm} of 44%.

Dewatering is of course only necessary when the particulate material is highly wet, depending on the weather conditions which were the one of the crop from which the particulate is issued
Step S5: Storage and fermentation are implemented preferably during a time sufficient so that the temperature T^{p} of the stored particulate material may increase initially and becomes stable (around 50° C.) or decreasing after depletion of nutrients and/or water. The total dry matter of the stored particulate material has a variation percentage per 24H, equal to or lower than, in an increasing order of preference, 20%; 15%; 10%; 5%; 1%.

Storage of the press cake from S4 or of the non-dewatered particulate material in large heaps or windrows lasts for up to 12-18 months. For example, the heaps or windrows are up to 5 m high and are stored over a period of at least 5 weeks.

A temperature T^{p} profile is regularly recorded at 1 m depth in the heap/windrow, using a probe. The temperature T^{p} profile can be e.g., as follows::8° C.; after 7 days: 12° C.; after 14 days: 15° C.; after 21 days: 30° C.; after 28 days: 45° C.; after 35 days: 45° C.; after 42 days: 44° C.

Step S5 results in a mass loss in a heap/windrow of approximately 30 m³ of the press cake from S4 (e.g. chopped and dehydrated corn stalks). The mass loss is determined as follows. Assuming, for instance:
a fresh mass of material, on the day of pressing, of 7500 kg, a dry matter content of 44%, and thus a total dry matter mass of 3300 k;
and a fresh mass of material, after 42 days from the day of pressing of 7162 kg, a dry matter content of 44%;
and thus a total dry matter mass of 3151 kg.

This results in a mass loss of 4.5% over the observed storage period of 42 days. This loss results in an O₂ consumption of approx. 51.4 g per kg dry matter or approx. 1.22 g O₂ per kg dry matter and day. At the end of 20 days, the O₂ consumption is clearly <1.5 g O₂ per kg dry matter and day, i.e., well within the “storage stable” designation according to the publication Gomez, Lima and Ferrer, published in Waste management & research 24 (1), 37-47, 2006.
Step S6: Grinding or milling, in particular to defibrate the stalks particles of the material from S5. It is for instance carried out with an impact mill, type Huning HPZ1200.
Step S7: Water spraying of particulate material from S6 is implemented by any conventional spraying means.
.S14. Step S8: Possible dewatering in the same way as in step S4, so as to get a dry matter content C^{dm}, in % by mass, such as:
   C^{dm}≤37, preferably C^{dm}≤35, and more preferably 20≤C^{dm}≤35.
Step S9: Loosening of the dewatered particulate material from (S8) in hammer mill, type Agerskov AM110.
Step S10: Possible addition of at least one filler, preferably of clay, type montmorillonite, in an amount comprised between e.g. 5% and 20% by dry mass.
   Standard casings, notably all peat based casings, can be also incorporated as fillers/additives.
Step S11: Possible fermenting the particulate material, during a time sufficient so that the temperature of the fermented particulate material reaches a value T^{p} greater than or equal to, in ° C. and in an increasing order of preference; 45; 50; 55; 60; 65.

Microbiological analysis of this material showed no presence of human pathogens (Enterobacteriaceae, E. coli, Campylobacter ssp., Listeria monocytogenes, Salmonella ssp) and/or potential diseases of Agaricus (Mycogone sp, Lecanicilium fungicola, Cladobotryum ssp). In addition, the material has no detectable levels of pesticides or plant growth regulators. This means that the material is safe for commercial utilization. However, the material may contain spores of Coprinus and Peziza, which may develop under industrial Agaricus production conditions, depending on the blend used, wet storage time, irrigation procedure, and more.

In a remarkable way of implementation of the method according to the invention, the fermentation S8 is done in an insulated container, in order to secure homogenous temperature T^{p} as above defined. The surface temperature of the mass (heap) of particulate material to be fermented is also controlled through this way of implementation.

This makes it possible to obtain a fermented material used as casing, which does not contain relevant levels of unwanted fungal spores
Step S12: Possible maintaining the fermented particulate material at the temperature T^{p} during at least, in hours and according to an increasing order of preference, 12; 24; 36; 48.
Possible Step S13: Possible storing and/or conditioning the fermented particulate material from .S12. For instance, it could be packed in big bags.

The water holding volumes of the fibrous fermented material so produced, are tested in several variants. The formation of agglomerates is stimulated in a mixing process similar to the usual casing in the mushroom industry. It is confirmed that thanks to the formation of the agglomerates and the capillary water binding, the water content can be increased from 330 g/l to >600 g/l (without clay) or >800 g/l (with clay and/or peat as additives). An average fiber length of 1-1.5 mm is preferred for the fibrous fermented material so produced.

Moreover, the addition of clay in mixtures without standard casing significantly improves the strength and thus ensures the industrial suitability of the casing.

### Variant of Implementation: Steps S01 to S012

Step S01: Implementing cut stalks of monocotyledonous flowering plants, preferably maize.
Step S02: shredding the cut stalks from (S01) into a particulate material using a????, with a cutting length of e.g. 2-8 mm to get a chopped particulate material.

The following steps are identical to steps 04-12 of the main mode of implementation.

### EXAMPLES

### Example 1

Step S1: The grains of the raw material corn (Zea mays) are harvested using a Claas Lexion®, a standard combine harvester. The varieties used were Pioneer® 0725, Pioneer® 0312,

Dekalb® 4598 and Dekalb® 5141.
Step S2: The harvest of the stems is conducted using a Claas Jaguar® forage harvester. This harvest is executed 2 days after the grain harvest, at an average dry matter content of the material of 27%. Cutting is executed using a direct disc mower, for a cutting length of around 5 mm.
Step S3: The harvested material is delivered to a processing site.
Step S4: It is subjected to a mechanical dewatering of the residual moisture and dissolved nutrients. Dewatering is executed using a Trumag® EX40 screw press. The dry matter content of the resulting press cake is 43%. The press juice is collected and redistributed to the agricultural fields.
Step S5: The press cake is put into an aerated and insulated composting container. It has a starting volume of around 48 m³, a total mass of 12.0 t, a dry matter content of 43% and a total dry matter of 5.16 t. After 4 weeks, the material has a temperature T^{p} of 52° C., no apparent volume loss, and total dry matter of 4.92 t. After 6 weeks, there is no more change in the temperature and the total dry matter of the material.
Step S6: After 6 weeks, the material is mechanically disintegrated using a Huning® HPZ1200 impact mill, with a retaining floor allowing for longer residence time of the material inside the milling chamber. This machine is set to maximize the friction and disintegration efficiency:
Step S7: After disintegration, the material is sprayed with water. The water addition is around 150 L per m³ of material throughput.
Step S8: The watered material of step S7 is subjected to water extraction. Dewatering is executed using a Trumag® EX40 screw press. The dry matter content of the resulting press cake is 43%. The press juice is collected and redistributed to the agricultural fields.
Step S9: The press cake is loosened in structure using a rotating mill AGERSKOV KM110.
Step S10 and/or S13: The so produced press cake is used for preparation of several blends 1-6 (see Table 1 below). Blend 4 is prepared using semi-dry milled black peat with pH 4. Blends 5 and 6 are prepared using standard, black peat based casing with corrected pH.

### Measurements

The density of materials is measured by filling a 20 L cylinder for density determination according to EN12580.

The material dimensions are measured using the method explained above.

Dry matter content was measured as dry mass as determined according to the method described above.

Total water content is the difference between fresh mass and dry mass.

Capillary bonded water is determined by filling the material at water saturation into a cylinder with a perforated bottom and subjecting the material to a suction pressure of 0.1 bar from below (standard procedure https://www.vegetronix.com/TechInfo/How-To-Measure-Holding-Capacity-Soil for determination of the field capacity: https://www.vegetronix.com/TechInfo/How-To-Measure-Holding-Capacity-Soll.

The water passing through the filter is capillarily bonded. The matrically bonded water is calculated as the difference between total water and capillary water.

The stability of the agglomerates was determined by the so-called “snow ball test”, which is a well introduced test in the industry. The material at water saturation is used to form a snow ball manually and this snow ball is thrown against a wall. If the entire snow ball sticks to the wall and no parts fall to the ground, the material is sticky and the agglomerates of good stability.

### Assessment of the so Obtained Fermented Materials

### Results

Table 1 shows the differences between the 6 tested blends. From a practical viewpoint, the total water and the stability of the agglomerates are most important. The blend with finer fibers and clay offers a very high total water content of 620 g/L. The specific water content and the total available water for growth of the fruit bodies are important for the mushroom yield. The total available water for growth of the fruit bodies is estimated to include all of the capillary water plus a part of the matrical water.

TABLE 1

| | | | | dry matter | total | matrical | capillary | specific |
|---|---|---|---|---|---|---|---|---|
| | | | density* | content | water | water*** | water** | water |
| nr | definition | structure | g/l | g/l | g/l | g/l | g/l | g/g |
| 1 | Corn fibres 2 mm, | loose | 400 | 70 | 330 | 280 | 50 | 0.83 |
| | 100% v/v | | | | | | | |
| 2 | Corn fibers 1 mm, | agglomerated, | 650 | 120 | 530 | 280 | 250 | 0.82 |
| | 100% v/v | weak | | | | | | |
| 3 | Corn fibers nr. | agglomerated, | 780 | 160 | 620 | 300 | 320 | 0.79 |
| | 2 + 40 g/l clay | strong | | | | | | |
| 4 | Corn fibers nr. | agglomerated, | 780 | 160 | 620 | 300 | 320 | 0.79 |
| | 2 80% v/v + | strong | | | | | | |
| | 20% milled peat + | | | | | | | |
| | 40 g/l clay | | | | | | | |
| 5 | Corn fibers nr. | agglomerated, | 800 | 200 | 600 | 350 | 250 | 0.75 |
| | 2 50% v/v + | strong | | | | | | |
| | black peat | | | | | | | |
| | casing 50% v/v | | | | | | | |
| 6 | Corn fibers nr. | agglomerated, | 830 | 220 | 610 | 380 | 230 | 0.73 |
| | 2 50% v/v + | strong | | | | | | |
| | black peat | | | | | | | |
| | casing 50% v/v + | | | | | | | |
| | 20 g/l clay | | | | | | | |

*at water saturation, determination as described above
**determination as described above
***calculated by difference of total water − capillar water

The variants were tested for mushrooms production for assessment of the mushrooms yield and quality. The fermented materials according to the invention with clay and/or peat meet all the technical specifications of a casing soil and are equivalent in yield and quality to 100% peat-based casing soil.

### Example 2

Step S1: The grains of the raw material corn (Zea mays) are harvested using a Claas Lexion, a standard combine harvester.
Step S2: The harvest of the stems is conducted using a Claas Jaguar forage harvester. This harvest is executed 2 days after the grain harvest, at an average dry matter content of the material of 27%. Cutting is executed using a direct disc mower, for a cutting length of around 5 mm.
Step S3: The harvested material is delivered to a processing site.
Step S4: It is subjected to a mechanical dewatering of the residual moisture and dissolved nutrients. Dewatering is executed using a Trumag EX40 screw press. The dry matter content of the resulting press cake is 43%. The press juice is collected and redistributed to the agricultural fields.
Step S5: The press cake is put into an aerated and insulated composting container. It has a starting volume of around 48 m³, a total mass of 12.0 t, a dry matter content of 43% and a total dry matter of 5.16 t. After 4 weeks, the material has a temperature of 52° C., no apparent volume loss, and total dry matter of 4.92 t. After 6 weeks, there is no more change in the temperature and the total dry matter of the material.
Step S6: After 6 weeks, the material is mechanically disintegrated using a Huning HPZ1200 impact mill, with a retaining floor allowing for longer residence time of the material inside the milling chamber. This machine is adapted to maximize the friction and disintegration efficiency:
Step S7: After disintegration, the material is sprayed with water. The water addition is around 150 | per m³ of material throughput.
Step S8: The watered material of step S7 is subjected to water extraction. Dewatering is executed using a Trumag® EX40 screw press. The dry matter content of the resulting press cake is 43%. The press juice is collected and redistributed to the agricultural fields.
Step S9: The press cake is loosened in structure using a rotating mill.
Step S10: Clay is added in a ratio of 25 kg per m³ of fibre material and blended using a Strautmann® Vertimix® 500 fodder blender with a vertical screw.

Steps $11 & S12: This blend is put into the aerated and insulated composting container. It reaches a temperature T^{p} of 67° C. within 60 hours and is kept at that temperature level for another 48 hours. At this point, the material is pasteurized and ready for industrial use.

The material forms stable agglomerates with the following specifications:
Greatest dimension D^{g} of the particles: 2 mm.
Specific density D^{s}: 161 g dry matter per L.
Specific water content C^{w}: 833 g per I.
C/N=102.55.
average length L^{a} of the fibers particles=1 mm.
No development of unwanted fungi is observed.

1. Fermented material comprising stalk particles of monocotyledonous flowering plants, preferably maize, and, preferably, at least one filler/additive, wherein:
(a) the particles have a size given by their greatest dimension D^{g}, in % passage through a 1.5 mm screen at dry stage, and in an increasing order of preference, such as:
$${D}^{g} > 20 ; {D}^{g} > 30 ; {D}^{g} > 40 ; {D}^{g} > 50$$
(b) the specific density D^{s} of the material, in g dry matter per liter and in an increasing order of preference, such as:
$$100 \leq {D}^{s} \leq 250 ; 120 \leq {D}^{s} \leq 220 ; 130 \leq {D}^{s} \leq 200 ; 140 \leq {D}^{s} \leq 200$$
(c) the specific water content C^{w} at saturation of the material is, in % by mass and in an increasing order of preference, such as:
$$55 \leq {C}^{w} \leq 95 ; 65 \leq {C}^{w} ; 70 \leq {C}^{w} \leq 90 ; 80 \leq {C}^{w} \leq 85$$
(d) the Carbon/Nitrogen ratio C/N of the material is, in an increasing order of preference, such as:
$$60 \leq C / N ; 70 \leq C / N ; 80 \leq C / N ; 90 \leq C / N ; 95 \leq C / N \leq 120 .$$

2. Fermented material according to claim 1 wherein at least a part of the particles are fibers particles, the average length L^{a} of the fibers particles being preferably, in mm and in an increasing order of preference, such as:
$$0.5 \leq {L}^{a} \leq 3 ; 0.6 \leq {L}^{a} \leq 2.5 ; 0.7 \leq {L}^{a} \leq 2 ; 0.8 \leq {L}^{a} \leq 1.8 ; 1 \leq {L}^{a} \leq 1.5 ;$$

3. Fermented material according to claim 1 having a biological stability corresponding to an oxygen consumption O^{c} lower than or equal, in g per day and g of material and in an increasing order of preference: 3; 2.8; 2.7; 2.6; 2.5; 2.4; 2.3; 2.2; 2.1; 2.

4. Fermented material according to claim 1 characterized in that it is a culture medium and/or plant substrate component and/or a peat substitute.

5. Fermented material according to claim 1 characterized in that it is mixed with standard casings in a proportion preferably of up to 70% v/v, preferably 5-40% v/v.

6. A method for the production in particular of the fermented material according to claim 1, comprising the following steps:
.S1. harvesting the grains of monocotyledonous flowering plants, preferably from the subfamily Panicoideae, and more preferably maize, so as the stalks remain stand on the field;
.S2. cutting and, preferably shredding, the remaining stalks standing on the field into a particulate material;
.S3. possible transporting the particulate material to a processing site;
.S4. possible dewatering the particulate material up to a dry matter content Com, in % by mass and in an increasing order of preference, such as:
$$20 \leq {C}^{d m} ; 25 \leq {C}^{d m} \leq 60 ; 30 \leq {C}^{d m} \leq 50$$
.S5. storing and submitting the particulate material, to a fermentation, preferably during a time sufficient so that the temperature T^{p} of the stored particulate material reaches a value around 50° C., and the total dry matter of the particulate material has a variation percentage per 24H, equal to or lower than, in an increasing order of preference, 20%; 15%; 10%; 5%; 1%;
.S6. grinding the particulate material up to a particle size given by their greatest dimension D^{g}, in mm and in an increasing order of preference, such as:
$${D}^{g} \leq 30 ; {D}^{g} \leq 20 ; {D}^{g} \leq 15 ; {D}^{g} \leq 10 ; {D}^{g} \leq 5$$
.S7. water spraying on the grinded particulate material from (S6);
. S8. possible dewatering the grinded particulate material, so as to get a dry matter content C^{dm}, in % by mass, such as:
C^{dm}≤37, preferably C^{dm}≤35, and more preferably 20≤C^{dm}≤35;
.S9. loosening of the particulate material;
.S10. possible addition of at least one filler/additive, preferably consisting in at least one clay;
.S11. possible submitting the particulate material, to a fermentation during a time sufficient, so that the temperature of the fermented particulate material reaches a value T^{p} (° C.) greater than or equal to, in an increasing order of preference; 50; 55; 60; 65;
.S12. possible maintaining the fermented particulate material at the temperature T^{p} during at least, in hours and according to an increasing order of preference, 12; 24; 36; 48;
.S13. possible storing and/or conditioning the fermented particulate material from .S12.

7. A method according to claim 6 wherein the steps .S1.; S2. & .S3. are replaced by the following steps:
.S01. implementing cut stalks of monocotyledonous flowering plants, preferably from the subfamily Panicoideae, and more preferably maize;
.S02. shredding the cut stalks from (S01) into a particulate material;
.S03. possible storing of the particulate material.

8. A method according to claim 6, wherein step (S2) or step (S02) is implemented by means of forage harvester.

9. A method according to claim 6 wherein step (S2) or (S02) leads to stalk particles having a particle size given by their greatest dimension D₂^{g}, in mm and in an increasing order of preference, such as:
$${D}_{{2}^{g}} \leq 15 ; {D}_{{2}^{g}} \leq 12 ; {D}_{{2}^{g}} \leq 9 ; {D}_{{2}^{g}} \leq 6 ; {D}_{{2}^{g}} \leq 3$$

10. A method according to claim 6 wherein step (S4) is a mechanical dewatering implemented by means of a screw press.

11. A method according to claim 6 wherein:
step (S5) of composting consists in loading the particulate material in an aerated and insulated composting container;
step (S11) of composting consists in loading the particulate material, in an aerated and insulated composting container.

12. A method according to claim 11 wherein the aerated and insulated composting container is a modified refrigerated shipping container.

13. A method according to claim 6 wherein step (S6) is a grinding implemented by means of an impact mill or of a grinding mill, preferably by means of an impact mill and more preferably by means of an impact mill which tip speed of the rotating working tools is greater than or equal to, in m/s and in an increasing order of preference; 60; 65; 70; 75; and more preferably comprised between 80 and 100.

14. A method according to claim 6 wherein in step (S7), the addition of water is comprised between 100 and 200 L/m³, preferably between 130 and 170 L/m³, and more preferably between 145 and 155 L/m³.

15. A method according to claim 6 wherein in step (S10)), the addition of filler is comprised between 5 and 45 kg/m³, preferably between 15 and 35 kg/m³, and more preferably between 20 and 30 kg/m³.
